SEQUENCE LISTING (23 sequences)

SEQ ID NO: 1 - protein, 36 aa, B. pertussis
TIRRGDAPAGGAVPGGAVPGGAVPGGFGPGGFGPVL

SEQ ID NO: 2 - protein, 10 aa, B. pertussis
GGFGPGGFGP

SEQ ID NO: 3 - protein, 10 aa, B. pertussis
GGAVPGGAVP

SEQ ID NO: 4 - protein, 20 aa, B. pertussis
GGAVPGGAVPGGFGPGGFGP

SEQ ID NO: 5 - protein, 15 aa, B. pertussis
GGAVPGGFGPGGFGP

SEQ ID NO: 6 - protein, 910 aa, B. pertussis
MNMSLSRIVKAAPLRRTTLAMALGALGAAPAAHADWNNQSIVKTGERQHGIHIQGSDPGGVRTASGTTIKVSGRQAQGILLENPAAELQFRNGSVTSSGQLSDDGIRRFLGTVTVKAGKLVADHATLANVGDTWDDDGIALYVAGEQAQASIADSTLQGAGGVQIERGANVTVQRSAIVDGGLHIGALQSLQPEDLPPSRVVLRDTNVTAVPASGAPAAVSVLGASELTLDGGHITGGRAAGVAAMQGAVVHLQRATIRRGDAPAGGAVPGGAVPGGAVPGGFGPGGFGPVLDGWYGVDVSGSSVELAQSIVEAPELGAAIRVGRGARVTVSGGSLSAPHGNVIETGGARRFAPQAAPLSITLQAGAHAQGKALLYRVLPEPVKLTLTGGADAQGDIVATELPSIPGTSIGPLDVALASQARWTGATRAVDSLSIDNATWVMTDNSNVGALRLASDGSVDFQQPAEAGRFKVLTVNTLAGSGLFRMNVFADLGLSDKLVVMQDASGQHRLWVRNSGSEPASANTLLLVQTPLGSAATFTLANKDGKVDIGTYRYRLAANGNGQWSLVGAKAPPAPKPAPQPGPQPPQPPQPQPEAPAPQPPAGRELSAAANAAVNTGGVGLASTLWYAESNALSKRLGELRLNPDAGGAWGRGFAQRQQLDNRAGRRFDQKVAGFELGADHAVAVAGGRWHLGGLAGYTRGDRGFTGDGGGHTDSVHVGGYATYIADSGFYLDATLRASRLENDFKVAGSDGYAVKGKYRTHGVGASLEAGRRFTHADGWFLEPQAELAVFRAGGGAYRAANGLRVRDEGGSSVLGRLGLEVGKRIELAGGRQVQPYIKASVLQEFDGAGTVHTNGIAHRTELRGTRAELGLGMAAALGRGHSLYASYEYSKGPKLAMPWTFHAGYRYSW

SEQ ID NO: 7 - DNA, 26 bp, Bordetella pertussis
cgggatccacgatacggcgcggggac

SEQ ID NO: 8 - DNA, 26 bp, Bordetella pertussis
gctctagagaggacgggaccgaagcc

SEQ ID NO: 9 - protein, 15 aa, B. pertussis
ATIRRGDAPAGGAVP

SEQ ID NO: 10 - protein, 15 aa, B. pertussis
GDAPAGGAVPGGAVP

SEQ ID NO: 11 - protein, 15 aa, B. pertussis
GGAVPGGAVPGGAVP

SEQ ID NO: 12 - protein, 15 aa, B. pertussis
GGAVPGGAVPGGFGP

SEQ ID NO: 13 - protein, 15 aa, B. pertussis
GGAVPGGFGPGGFGP

SEQ ID NO: 14 - protein, 15 aa, B. pertussis
GGFGPGGFGPVLDGW

SEQ ID NO: 15 - protein, 15 aa, B. pertussis
GGFGPVLDGWYGVDV

SEQ ID NO: 16 - protein, 15 aa, B. pertussis
GGFGPGGFGPGGFGP

SEQ ID NO: 17 - protein, 16 aa, Neisseria meningitidis
TPAYYTKDTNNNLTLV

SEQ ID NO: 18 - protein, 16 aa, Neisseria meningitidis
TPAYYTKETNNNLTLV

SEQ ID NO: 19 - protein, 36 aa, Bordetella pertussis; identical to SEQ ID NO: 1

SEQ ID NO: 20 - protein, 41 aa, Bordetella pertussis
TIRRGDAPAGGAVPGGAVPGGFGPGGFGPGGFGPGGFGPVL

SEQ ID NO: 21 - protein, 36 aa, Bordetella pertussis
TIRRGDAPAGGAVPGGAVPGGFGPGGFGPGGFGPVL

SEQ ID NO: 22 - protein, 31 aa, Bordetella pertussis
TIRRGDAPAGGAVPGGAVPGGFGPGGFGPVL

SEQ ID NO: 23 - protein, 36 aa, Bordetella pertussis
TIRRGDAPAGGGVPGGAVPGGFGPGGFGPGGFGPVL

(19) United States
(12) Patent Application Publication
(10) Pub. No.: US 2003/0187221 A1
(43) Pub. Date: Oct. 2, 2003

(54) Peptides for the preparation of vaccines against bordetella pertussis and bordetella parapertussis

(76) Inventor: Frederik Robert Mooi, Wijk Bij Duurstede (NL)

Correspondence Address: YOUNG & THOMPSON, 745 SOUTH 23RD STREET 2ND FLOOR, ARLINGTON, VA, 22202

(21) Appl. No.: 10/312,732

(22) PCT Filed: Jun. 29, 2001
(86) PCT No.: PCT/NL01/00493

(30) Foreign Application Priority Data
Jun. 30, 2000 (EP) 00202309.1

(51) Int. Cl. C07K 1/00; C07K 14/00; C07K 17/00

(52) U.S. Cl. 530/350

(57) ABSTRACT

The present invention discloses polypeptides for use in vaccines against pertussis and parapertussis. The polypeptides of the invention are derived from the Bordetella pertussis pertactin. Preferably, the polypeptides of the invention comprise sequences from region 1 of pertactin, in particular they comprise one or more “GGFGP” or “GGAVP” repeats. Alternatively the polypeptides of the invention have a pertactin sequence from which part or all of region 1 or region 2 is deleted. The polypeptides of the invention are incorporated in compositions that are useful as vaccines for human and veterinary purposes. The invention further discloses antibodies against the pertactin-derived polypeptides of the invention.

### FIELD OF THE INVENTION

The present invention relates to peptides or polypeptides that can be used for pharmaceutical and/or veterinary purposes, and in particular in the preparation of vaccines against Bordetella pertussis and/or Bordetella parapertussis (henceforth collectively referred to as “Bordetella (para)pertussis”.) The invention also relates to vaccines containing such peptides, and to the use of such peptides in the preparation of vaccines. The invention further relates to antibodies generated against these peptides, and to pharmaceutical compositions containing such antibodies.

### BACKGROUND OF THE INVENTION

Conventionally, vaccines against pertussis (“whooping-cough”) have been based on whole cells of B. pertussis. In recent years, besides these so-called “whole cell vaccines” or “WCV's”, also vaccines have been developed that contain specific antigens derived from B. pertussis, such as fimbriae, filamentous hemagglutinin, pertussis toxin and/or a surface-associated protein of B. pertussis referred to as “pertactin”. These so-called “acellular vaccines” or “ACVs” have now been introduced in several countries.

The sequence of B. pertussis pertactin has been published (vide inter alia Charles et al., Proc. Natl. Acad. Sci. USA May 1989; 86(10): 3554-8 and SEQ ID NO: 6 below). Pertactin is a 69 kD protein comprising approximately 926 amino acid residues, which from X-ray data is known to form a helix with several protruding loops that contain sequence motifs associated with the biological activity of the protein (Emsley at al., Nature (1996) 381:90-92). These include a loop containing an Arg-Gly-Asp (RGD) tripeptide motif (amino acids 260-262) involved in adherence to host tissues and a loop with a (PQP)₅ motif near the carboxy- terminus containing the a major immunoprotective epitope (Charles et al., Eur. J. Immunol. (1991) 21:1147-1153.).

It is also known that pertactin is formed from a precursor, which is encoded by a gene referred to as “prn”. This gene, which is schematically shown in FIG. 1, comprises two regions, referred to as “region 1” and “region 2” respectively (vide also Mooi et al., Infect Immun. (1998) 66:670-675). Of these, region 1 flanks the RGD motif and comprises a number of GGxxP repeats, whereas region 2 comprises the repeated PQP motif mentioned above.

Although WCV's have been very successful in the past and are still very effective in preventing B. pertussis infections, there are signs that pertussis may be reemerging, also in Western countries with long traditions in pertussis vaccination, such as the United States, the Netherlands, Canada and Australia ( Mooi et al., Infect. Immun. (1999) 67:3133-3134). The present inventors have already proposed one explanation for this phenomenon: antigenic divergence between vaccine strains and circulating strains, in particular with respect to pertactin.

In support of this explanation, the present inventors have also shown that the prn gene that encodes the pertactin precursor is polymorphic. So far, up to 9 different pertactin types have been identified in B. pertussis strains circulating in the world. It was also shown that variation between these pertactin types is mainly restricted to region 1, and consists of deletions or insertions in the repeat unit GGxxP of region 1. Reference is again made to Mooi et al. (1998, 1999, supra); and Mastrantonio et al., Microbiology (1999) 145: 2069.

Besides B. pertussis, about 1 to 5 percent of pertussis cases is caused by (strains of) B. parapertussis. In this respect, however, there is some evidence that ACV's based upon B. pertussis antigens—including ACV's based upon intact B. pertussis pertactin—may not (also) provide sufficient protection against (strains of) B. parapertussis. For instance, tests in a mouse model have shown that immunization with intact B. pertussis pertactin does not provide protection against B. parapertussis (Khelef et al., Infect. Immun. 1993; 61(2); 486-90). Thus, it may be that the introduction of this novel generation of ACV's may in future lead to a relative increase in whooping cough cases that are caused by B. parapertussis.

Since the pertussis vaccines currently used may not be equally effective in providing protection against the different circulating strains of B. pertussis, and may not be effective in providing protection against (strains of) B. parapertussis, the development of novel vaccines against B. (para)pertussis, and of antigens that may be used in the preparation of such vaccines, remains an ongoing concern, in order to ensure the long term efficacy of pertussis vaccines.

### DESCRIPTION OF THE INVENTION

Accordingly, it is a general object of the invention to make available a vaccine that can be used to provide immunity against essentially all strains of B. pertussis, or at least against essentially all strains of B. pertussis that are currently known to circulate in the countries mentioned above, and in particular in Europe. Preferably, however, such a vaccine would also be able to provide protection against (all) other strains of B. pertussis, including but not limited to those strains that are yet to be identified and/or that may arise in future. Most preferably, such a vaccine should also provide protection against (stains of) B. parapertussis, or at least provide a degree of protection against (strains of) B. parapertussis that is better than the protection provided by the current generation ACV's based upon B. pertussis antigens, and in particular upon essentially intact B. pertussis pertactin.

In particular, it is an object of the invention to provide a pharmaceutical and/or veterinary component that, upon administration to a human being, preferably an infant, or another mammal, can provide protection against B. (para)pertussis, e.g. by generating a protective immune response in said human being or mammal. As will be clear to the skilled person, such a component could be used as an antigenic component in the preparation/formulation of vaccines against pertussis, both for human or veterinary application, including but not limited to combination vaccines known per se, such as “DTP” vaccines.

Surprisingly, it has now been found that the above objects can be achieved by the use of a peptide derived from “region 1” of B. pertussis pertactin, and in particular by the use of a “peptide of the invention” as defined herein below. Inter alia, this finding is particularly surprising because—whereas the use of an intact pertactin protein (e.g. as present on a B. pertussis cell in an WCV or as used as an antigen in an ACV) in practice does not lead to a vaccine that is equally efficacious against the different strains of B. pertussis—it has now been found that such cross-immunity can even be obtained by using a vaccine based upon a small protein derived from said pertactin. Also, it is expected that—despite the differences in the pertactin of B. pertussis and the pertactin of B. parapertussis—the vaccines of the invention will also provide at least some degree of protection against (strains of) B. parapertussis, e.g. better than the protection provided by the current generation of ACV's.

This finding is even more surprising because—as mentioned above—these strains of B. pertussis differ in the distinct pertactin types as expressed on their surface, and because between these distinct types, variation is essentially restricted to “region 1”, i.e. the same amino acid sequence/epitope from which the peptide(s) used in the present invention are now derived.

It is conceivable that infection or vaccination mainly results in the induction of antibodies directed against conformational epitopes of pertactin. In this context it is significant that monoclonal antibodies directed against conformational epitopes in region 1 were type specific, whereas those that recognize a linear epitope bound to all B. pertussis pertactin variants. Thus, to prevent cross-immunity, the bacteria may have evolved mechanisms to direct the immune response towards conformational epitopes. Extending this line of reasoning, it is plausible that the vaccination with a peptide derived from region 1 will induce cross-protective antibodies, as they will be directed against a linear epitope. It may also be that—in contrast to antibodies generated against conformational epitopes of B. pertussis pertactin—such antibodies against a linear epitope of B. pertussis pertactin can also bind to B. parapertussis pertactin.

However, it should be noted that the present invention is not limited to any specific explanation of, nor to any mechanism for, the efficacy of the peptides described hereinbelow, nor for the way in which these peptides provide their advantageous cross-immunity.

Thus, in a first aspect, the invention relates to a peptide that:

(a) comprises between 6 and 40, and in particular between 10 and 15, contiguous amino acid residues from the amino acid sequence of “region 1” of a B. pertussis pertactin; and that

(b) preferably comprises a total of between of between 6 and 100 amino acid residues, and in particular of between 6 and 50 amino acid residues, more in particular between 10 and 40 and amino acid residues.

In particular, the invention relates to a peptide that:

a) comprises between 6 and 36, and in particular between 10 and 15, contiguous amino acid residues from the amino acid sequence of SEQ ID NO: 1:

TIRRGDAPAGGAVPGGAVPGGAVPGGFGPGGFGPVL (SEQ ID NO: 1);

and that

b) preferably comprises a total of between of between 6 and 100 amino acid residues, and in particular of between 6 and 50 amino acid residues, more in particular between 10 and 50 amino acid residues.

The peptides mentioned above will also be referred to hereinbelow as “peptides of the invention”. The above other amino acid sequence specified herein will be given in the standard “one letter amino acid code”.

Usually, the peptides of the invention will consist essentially only of amino acid residues derived from/corresponding to the amino acid residues of region 1 of a Bordetella pertussis pertactin, and thus contain a total of between 6 and 40, preferably between 10 and 15 amino acid residues. However, as will be clear from the definitions given hereinabove and also from the further description hereinbelow, the presence of some additional amino acid residues is not excluded, e.g. to a total of 100 amino acid residues, preferably 50 amino acid residues.

These additional amino acid residues may for instance correspond to one or more amino acid residues of the sequence of pertactin that lie “outside” region 1. For instance, these may be amino acid residues that in the sequence of pertactin lie adjacent to (the sequence of) region 1, e.g. the first 20, and preferably 10, amino acid residues that lie directly preceding and/or directly following (the sequence of) region 1.

However, the invention is in its broadest sense is not limited to the further amino acid residues that may be present (i.e. additional to the amino acids of region 1); as long as the further requirements outlined herein are met In addition, it should also be noted that—as outlined above—“analogues” of the peptides of the invention may be used, including but not limited to analogues into which one or more amino acid residues have been inserted, and or to which one or more amino acid residues have been added. Also, as described below, the peptides of the invention may be provided/used as a protein fusion with at least one amino acid sequence with which the peptide of the invention is not naturally associated ( i.e. in (a) native pertactin).

The invention also relates to a composition, preferably a pharmaceutical composition and in particular to a vaccine, that comprises at least one peptide of the invention, optionally in combination with at least one pharmaceutically acceptable carrier, excipient or adjuvant. Such a vaccine will also be referred to herein as a “vaccine of the invention”.

In another aspect, the invention relates to the use of a peptide of the invention in the preparation of a vaccine, and in particular in the preparation of a vaccine against B. (para)pertussis and optionally against one or more other infectious diseases of a human being, including but not limited to diphtheria, tetanus, polio and/or Haemophilus influenzae b (“Hib”).

The invention also relates to an antibody specific for, and/or generated against a peptide of the invention, and to a pharmaceutical composition containing at least one such antibody, optionally in combination with at least one pharmaceutically acceptable carrier, excipients or adjuvant.

In yet another aspect, the invention relates to a protein fusion comprising at least one peptide of the invention, and to a pharmaceutical composition that comprises at least one such protein fusion, optionally in combination with at least one pharmaceutically acceptable carrier, excipients or adjuvant.

Preferably, a peptide of the invention is such that, upon administration to a human being in a suitable manner and in a suitable amount (e.g. as outlined below), it is capable of eliciting in said human being an immune response. More preferably, a peptide of the invention is such that, upon administration to a human being in a suitable manner and in a suitable amount, it is capable of eliciting in said human being a protective immune response, and in particular an immune response which protects said human being against infection by B. pertussis, and preferably also against infection by B. parapertussis. Also, a peptide of the invention is preferably such that, upon administration to a human being in a suitable manner and in a suitable amount, it is capable of eliciting in said human being a “detectable immune response”, by which is generally meant an immune response that leads to at least one detectable biological change, and in particular to at least one detectable immunological change, in said human being. E.g., such a detectable immunological change can involve the generation of antibodies against the peptide of the invention, against (essentially intact) pertain, and/or against (whole cells of) B. (para)pertussis, or any other detectable and/or measurable immune response. Techniques for determining whether a peptide of the invention can provide such a “protective immune response” and/or such a “detectable immune response” will be clear to the skilled person, and may be essentially the same as, or may be essentially analogous to, the methods described in the Examples below. E.g., such a technique may involve immunizing a mouse or another suitable animal (mammal) with a peptide of the invention, and then determining whether antibodies are raised against said peptide, e.g. essentially as outlined in the Section 2C of the Examples. Alternatively, an animal immunized with a peptide of the invention may be exposed to viable B. (para)pertussis cells, after which the susceptibility to infection is compared to animals which have not been immunized, e.g. by means of determining the degree of colonization of the trachea and/or the lungs, e.g. essentially as outlined in Section 2D of the Examples. Particular preferred are those peptides of the invention which in a such test provide at least 10%, preferably at least 30%, more preferably at least 50%, and even more preferably at least 70%, of the protection provided by the peptide of SEQ ID NO: 5 mentioned above, as may for instance be determined by the titers of antibodies raised (e.g. detemined essentially as outleed in the Section 2C of the Examples) and/or the degree of protection against infection provided (e.g. determined essentially as outlined in Section 2D of the Examples).

Preferred peptides of the invention will generally contain one or more repeats GGFGP and/or one or more repeats GGAVP, in which the total number of such repeats will be between 2 and 10. For example, such preferred peptides of the invention include, but are not limited to:

| | |
|---|---|
| GGFGP-GGFGP | (SEQ ID NO:2) |
| GGAVP-GGAVP | (SEQ ID NO:3) |
| GGAVP-GGAVP-GGFGP-GGFGP. | (SEQ ID NO:4) |

Most preferred in the invention is the peptide of SEQ ID NO: 5:

GGAVPGGFGPGGFGP (SEQ ID NO: 5).

Besides the above peptides of the invention, the invention also comprises (the use of) analogues of such peptides, by which is generally meant a peptide of the invention as defined above in which at most 8, preferably at most 4, more preferably at most two, and even more preferably only one amino acid residue has been deleted, replaced, added and/or substituted. Amino acid substitutions in the analogues are preferably conservative amino acid substitutions. Conservative amino acid substitutions refer to the interchangeability of residues having similar side chains. E.g., a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Thus, a conservative amino acid substitution is a substitution where an amino acid from a given group is replaced by another amino acid from that same group. Preferred conservative amino acids substitution groups are: valine-leucine-isoleucine, phenylalanine-tyrosine, lysine-arginine, alanine-valine, and asparagine-glutamine.

Preferably, the analogue is such that, upon administration to a human being in a suitable manner and in a suitable amount, it is capable of generating/eliciting in said human being an immune response. More preferably, the analogue is such that, upon administration to a human being in a suitable manner and in a suitable amount, it is capable of eliciting a protective immune response in the human being, and in particular an immune response which protects the human being against infection by B. pertussis, and preferably also against infection by B. parapertussis, as may be determined using one or more of the techniques referred to above. Also, the analogue is preferably such that, upon administration to a human being in a suitable manner and in a suitable amount, it is capable of eliciting in said human being a “detectable immune response”, which for an analogue may also include the generation against antibodies against the specific analogue used, and as again may be determined using one or more of the techniques referred to above. Particularly preferred are analogues of the preferred peptides of the invention as defined above. Especially preferred are analogues of the peptide of SEQ ID NO: 5. In the further description below, the analogues defined above should be considered as encompassed within the term “peptides of the invention”, unless indicated otherwise. Also, the peptides of the invention and/or analogues thereof may be (used) in the form of a pharmaceutically acceptable salt, and such salts should also be considered as encompassed within the term “peptides of the invention” as used hereinbelow.

The peptides of the invention may be provided in a manner known per se, including of the invention can be prepared in a manner known per se, for instance via peptide synthesis techniques involving the use of protected amino acids, carbodiimide chemistry and/or Fmoc chemistry, which may be carried out using automated equipment Other techniques include, but are not limited to, solid-phase peptide synthesis, for instance as described by Merrifield, J. Am. Chem. Soc., 85, 2149 (1963). These and other techniques will be known to the skilled person and/or are described in the standard handbooks.

The peptides of the invention can be used in the preparation of pharmaceutical compositions, and in particular in the preparation of vaccines. More in particular, the peptides of the invention can be used in the preparation of vaccines that are intended to protect a human being against infection by B. (para)pertussis. These may be vaccines that are intended to protect solely against pertussis (i.e. so-called “monovalent” or “monocomponent” vaccine), but may also be a combination vaccine (i.e. a so-called “polyvalent” or “polycomponent” vaccine) intended to protect against pertussis and at least one further infectious disease other than pertussis, including but not limited to diphtheria, tetanus, polio and/or Hib. Examples of such “combination vaccines” will be clear to the skilled person, and e.g. include, but are not limited to, “DPTa” vaccines. (Also, it will be clear to the skilled person that such (combination) vaccines are often used to immunize/protect infants and children. Thus, as used herein, the term “human being”, although not limited to any specific age, will in particular include infants and children.)

The peptides or vaccines of the invention will generally be administered in an amount and according to a regimen that will elicit an immune response in a human being, preferably a detectable immune response as mentioned above and most preferably a protective immune response. Such a regimen may involve a single administration or two or more administrations, e.g. one or more priming immunizations followed by one or more booster immunizations. Suitable regimens will be clear to the skilled person and may essentially be the same as the regimens currently used with known vaccines against pertussis and/or with known combination vaccines. Usually, the peptide of the invention will be administered in an amount of between 1 and 100 Ig, and in particular between 4 and 20 μg/dosis. Thus, a suitable regimen may for instance involve priming immunizations with a dose of 10 μg peptide of the invention, followed by further priming immunizations after 2, 3, 4 and 11 months with the same dose, and a booster administration after 4 years with the same dose. The peptides or vaccines of the invention may be administered in any suitable manner known per se, for instance orally, transdermally, subcutaneously, intramuscularly, intraperitoneally, intravenously or via the respiratory tract, of which—as will be clear to the skilled person—intramuscular administration is particularly preferred.

The vaccines of the invention may be formulated in any manner known per se—which will usually depend upon the intended route of administration—optionally using one or more pharmacologically acceptable carriers, excipients or adjuvants. Usually, the vaccine of the invention will be in a form intended and/or suitable for injection, such as a solution or suspension of the peptide(s) in water or in a physiological acceptable buffer or solution. In this respect, the use of the peptides of the invention may also make it possible to provide such vaccines in a form which can be reconstituted—for instance with water or a physiological acceptable buffer or solution—to provide a preparation suitable for injection, for instance in the form of a dried or freeze-dried powder.

A vaccine of the invention may be suitably packaged in a suitable holder or container, for instance in an ampoule, a bottle or a flask, optionally in combination a leaflet containing product information. Generally, a vaccine of the invention will contain the peptide(s) of the invention in an amount of between 1 μg and 100 μg, and preferably between 4 μg and 20 μg, usually in the form of a unit dose. The vaccines of the invention may also comprise one or more immunological adjuvants acceptable for use in vaccines, such as aluminium phosphate and/or aluminium hydroxide. These may be used in suitable amounts known per se, for instance in amounts known per se from the formulation of known ACV's. Also, a peptide of the invention may be conjugated with, (covalently) linked to, or otherwise attached to, a functional residue, such as maltose-binding protein, tetanus toxoid or another carrier or immuno-modulating and/or immuno-stimulating group. In addition, the vaccines of the invention may also contain one or more B. (para)pertussis antigens known per se, such as pertussis toxoid, filamenteous hemagglutinin, serotype 2 fimbriae and/or serotype 3 fimbriae, for instance in amounts known per se from acellular vaccines. Also, in order to provide a combination vaccine as mentioned above, a vaccine of the invention may also contain one or more further pharmaceutically acceptable components that, upon administration to a human being in a suitable amount, can protect said human being against at least one (infectious) disease other than pertussis, for instance diphtheria, tetanus, polio and/or Haemophilus influenza b. Usually, such a component will be a suitable antigen known per se, including e.g.:

an antigen providing protection against diphtheria such as diphtheria toxoid;

an antigen providing protection against tetanus such as tetanus toxoid,

an antigen providing protection against polio such as inactivated polio virus;

an antigen providing protection against Haemophilus influenza b.

or a suitable combination thereof However, it is envisaged that any suitable antigen(s) that may become available after the priority date of the present application may also be used. Such further antigens will generally be used in amounts that, upon administration to a human being according to a suitable regimen (e.g. as outlined above for the vaccines of the invention), will generate/elicit in said human being an immune response, and in particular a protective immune response and/or a detectable immune response, against the intended disease. Suitable amounts for each specific antigen will be clear to the skilled person, and will usually correspond to the amounts that are normally used in vaccines known per se. Some preferred but non-limiting combination vaccines of the invention may for instance comprise two or more of the following components in the amounts indicated:

| | |
|---|---|
| peptide of the invention: | between 4 μg and 20 μg; |
| pertusis toxoid: | between 5 μg and 50 μg; |
| filamentous hemagglutinin: | between 5 μg and 50 μg; |
| serotype 2 fimbriae: | between 2.5 μg and 50 μg; |
| serotype 3 fimbriae: | between 2.5 μg and 50 μg; |
| diptheria toxoid: | between 5 μg and 50 μg; |
| tetanus toxoid: | between 5 μg and 50 μg; and/or |
| Hib (Haemophilus influenzae type B) | between 2 μg and 10 μg poly- |
| | saccharide (RPR: poly-ribosyl ri- |
| | bitol phosphate). |

Again, such combination vaccines of the invention may be formulated and administered in a manner known per se, e.g. as outlined above. More generally, the peptide(s) of the invention can be used to replace whole cells B. pertussis in known WCVs and/or to replace some or all of the pertussis antigens—including but not limited to (essentially) intact pertactin—currently used in known ACV's. However, it is also envisaged that the small antigenic peptides of the invention may open up novel types of pertussis vaccines and/or novel ways of formulating and/or administering pertussis vaccines.

As mentioned above, the peptides and vaccines of the invention have the advantage—compared to the currently available pertussis antigens and vaccines—that they can provide—i.e. simultaneously—immunity against (at least) the different strains of B. pertussis used in the Examples below, despite the differences between the pertactin proteins associated with the surface of said strains, i.e. due to polymorphisms in the pertactin encoding prn-genes between said strains. More broadly, it is expected that the peptides of the invention can be used to provide immunity against all (other) strains of B. pertussis currently circulating, and also any stains that may yet be identified and/or that may arise in future; and may also provide protection against (strains of) B. parapertussis. In addition, the use of a small antigenic peptides—compared to the use of the whole cells and/or the antigenic components currently used—may also provide increased stability of the vaccine.

Other possible applications of the peptides of the invention include, but are not limited to use in in vitro assays and/or in biological assays.

In yet another aspect, the invention relates to (protein) fusion comprising at least one peptide of the invention, linked to at least one further amino acid sequence which is different from the amino acid sequence with which a peptide of the invention is (or may be) natively associated in a naturally occurring pertain. Such a further protein sequence may optionally be antigenic per se and/or comprise one or more further (antigenic) epitopes, i.e. to provide (protective) immunity against B. (para)pertussis and/or another infectious disease, and/or may be an immuno-modulating and/or immuno-stimulating amino acid sequence.

Such fusions may be provided in a manner known per se, such as expression of a nucleotide sequence encoding such a fusion in a suitable host organism—e.g. under the control of a suitable promoter operable in the host organism used—followed by isolation/purification of the fusion from the host organism and/or the culture medium. Suitable techniques for providing nucleotide sequences encoding such fusions, for (heterologous) expression thereof in a host organism, suitable host organisms (such as Escherichia coli or B. (para)pertussis) and promoters operable therein (such as T7 RNA polymerase promoter, lac promoter, or promoters derived from B. (para)pertussis, such as the promoter for Omp or filamentous hemagglutinin ), and techniques for isolating the fusion(s) thus expressed will be clear to the skilled person. Preferably, such a fusion is such that, upon administration to a human being in a suitable manner and in a suitable amount, it is capable of generating/eliciting in said human being a protective immune response (in particular against B. pertussis, and preferably also against infection by B. parapertussis) and/or a detectable immune response, e.g. as outlined above.

The fusions mentioned above may be used in the preparation of pharmaceutical compositions, and in particular vaccines—i.e. against B. (para)pertussis—essentially in the manner outlined above for the peptides of the invention.

In yet another aspect, the invention relates to an antibody directed/generated against a peptide of the invention. As used herein, the term antibody includes inter alia polyclonal, monoclonal, chimeric and single chain antibodies, as well as fragments (Fab, Fv, and Fa) and an Fab expression library. Furthermore, “humanized” antibodies may be used, for instance as described WO 98/49306. Such antibodies can be obtained in a manner known per se, such as those described in WO 95/32734, WO 96/23882, WO 98/02456, WO 98/41633 and/or WO 98/49306. E.g., polyclonal antibodies can be obtained by immunizing a suitable host such as a goat, rabbit, sheep, rat, pig, mouse or human with a peptide of the invention, optionally with the use of an immunogenic carrier (such as bovine serum albumin or keyhole limpet hemocyanin) and/or an adjuvant such as Freund's, saponin, ISCOM's, aluminium hydroxide or a similar mineral gel, or keyhole limpet hemocyanin or a similar surface active substance. After an immune response against the peptide of the invention has been raised (usually within 1-7 days), the antibodies can be isolated from blood or serum taken from the immunized animal in a manner known per se, which optionally may involve a step of screening for an antibody with desired properties, such as desired specificity or affidity, using known immunoassay techniques, for which reference is again made to for instance WO 96/23882.

Monoclonal antibodies may be produced using continuous cell lines in culture, including hybridoma and similar techniques, again essentially as described in the above cited references. Fab-fragments such as F(ab)₂, Fab′ and Fab fragments may be obtained by digestion of an antibody with pepsin or another protease, reducing disulfide-linkages and treatment with papain and a reducing agent, respectively.

The antibodies generated against the peptides of the invention may be used for pharmaceutical and/or diagnostic purposes. For instance, said antibodies may be used to prevent and/or treat infections with B. (para)pertussis, e.g. by administering said antibody in a suitable manner known per se—e.g. intravenously, intramuscularly, intranasally—and a suitable amount—e.g. between 25 and 1500 mg/kg body weight—to a human being suffering from, or at risk of; infection by B. (para)pertussis. For this purpose, the antibodies will generally be formulate as a pharmaceutical composition that comprises at least one antibody generated against a peptide of the invention, optionally in combination with at least one pharmaceutically acceptable carrier, excipients and/or adjuvant; and such compositions form a further aspect of the invention. In particular, such a composition will be in the form of a composition suitable for injection and/or infusion, comprising a solution/suspension of the antibody in water or a suitable physiological buffer or solution. Some other uses of antibodies generated against the peptides of the invention may include, but are not limited to and any other pharmaceutical and/or diagnostic applications of antibodies known per se.

In yet another aspect, the invention relates to a polypeptide having the amino acid sequence of a peon from which, compared to the sequence of said pertactin:

the amino acid residues that form the so-called “region 1” have been at least partly, and preferably essentially completely removed; and/or from which,

the amino acid residues that form the so-called “region 2” have been at least partly, and preferably essentially completely removed.

Such a pertactin from which (at least part of) region 1 and/or from which (at least part of) region 2 has been removed will also be referred to hereinbelow as a “deleted pertactin”. In this aspect, the “sequence of a pertactin” may be a naturally occurring (type of) B. pertussis pertactin, or may be an analogue or variant thereof, including but not limited to synthetic analogues or variants thereof. In particular, this aspect of the invention relates to such a “deleted pertactin” which has the amino acid sequence of pertactin as shown in SEQ ID NO: 6—or the amino acid sequence of a naturally occurring or synthetic analogue or variant thereof—from which:

at least 5, preferably at least 10, and up to all of the amino acid residues at positions 266-290 have been removed; and/or from which

at least 3, preferably at least 10, and up to all of the amino acid residues at positions 569-603 have been removed.

Even more preferred, this aspect of the invention relates to a such a deleted pertactin having the amino acid sequence of pertactin as shown in SEQ ID NO: 6—or the amino acid sequence of a naturally occurring or synthetic analogue or variant thereof—from which one or more amino acid residues are/have been removed (i.e. as outlined above) such that:

from the amino acid residues between positions 260 and 290 of SEQ ID NO: 6, at least one repeat “GGAVP” and/or at least one repeat “GGFGP” is/has been removed, and up to all such repeats “GGAVP” and/or “GGFGP” that are present between positions 260 and 290.

Preferably, a total of 2, 3, 4 or 5 repeats “GGAVP” and/or “GGFGP” is removed, optionally with one or more further amino acid residues present between positions 260 and 290, e.g. in the form of a contiguous sequence of amino acid residues comprising said repeats; and/or such that:

from the amino acid residues between positions 569 and 603 of SEQ ID NO: 6, at least one motif “PQP” is/has been removed, and up to all motifs “PQP” that are present between positions 569 and 603.

Preferably, 1,2,3, 4 or 5 motifs PQP are removed; optionally with one or more further amino acid residues present between positions 569 and 603, e.g. in the form of a contiguous sequence of amino acid residues comprising said PQP motifs.

Most preferably, the deleted pertactin has the amino acid sequence of pertactin as shown in SEQ ID NO: 6—or the amino acid sequence of a naturally occurring or synthetic analogue and/or variant thereof—from which the following amino acid residues have been removed:

the residues from Gly at position 266 up to and including Pro at position 290; or

the residues from Gly at position 270 up to and including Pro at position 285; and/or from which any of the following amino acid residues have been removed.

the residues from Ala at position 569 up to and including Ala at position 603; or

the residues from Pro at position 572 up to and including Gin at position 599; or

the residues from Pro at position 575 up to and including Pro at position 596; or

the residues from Ala at position 578 up to and including Glu at position 594; or

the residues from Pro at position 581 up to and including Pro at position 591; or

the residues from Gln at position 584 up to and including Pro at position 588

In addition to the one or more amino acid residues from region 1 and/or from region 2 mentioned above, also one or more of the amino acid residues flanking region 1 and/or region 2 may (also) be removed, e.g. from Thr at position 206 up to and including His at position 340; and/or from Asn at position 569 up to and including Gly at position 653. As mentioned above, the deleted pertactin may have been derived from the sequence of SEQ ID NO: 6, or from a naturally occurring and/or synthetic analogue or variant thereof, i.e. an analogue or variant in, from or to which one or more amino acid residues have been inserted, deleted, substituted (preferably as a “conservative” substitution) or added. Preferably, such an analogue or variant has a percentage amino acid sequence homology, or rather amino acid sequence identity of more than 30%, preferably 40%, more preferably 50%, even more preferably 60%, still more preferably 70%, yet more preferably more than 80%, and most preferably more than 90%.

“Amino acid sequence identity”, as known in the art, is a relationship between two or more polypeptide sequences as determined by comparing the sequences. In the art, “identity” also means the degree of sequence relatedness between polypeptide sequences as determined by the match between strings of such sequences. “Similarity” between two polypeptides is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Infomatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M. Stockton Press, New York, 1991; and Carillo, H., and Lipman, D., SIAM J. Applied Math, 48:1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12 (1):387 (1984)), BestFit, BLASTP, BLASTN, and FASTA (Altschul, S. F. et al., J. Mol. Biol. 215:403-410 (1990). The BLASTX program is publicly available from NCBI and other sources (BLAST Manual, Altschul, S., et al., NCBI NLMNIH Bethesda, Md. 20894; Altschul, S., et al., J. Mol. Biol. 215:403-410 (1990). The well-known Smith Waterman algorithm may also be used to determine identity. Preferred parameters for polypeptide sequence comparison include the following: 1) Algorithm: Needleman and Wunsch, J. Mol. Biol. 48:443-453 (1970) Comparison matrix: BLOSSUM62 from Hentikoff and Hentikoff, Proc. Natl. Acad. Sci. USA. 89:10915-10919 (1992) Gap Penalty: 12; and Gap Length Penalty: 4. A program useful with these parameters is publicly available as the “Ogap” program from Genetics Computer Group, located in Madison, Wis. The aforementioned parameters are the default parameters for peptide comparisons (along with no penalty for end gaps).

Thus, a “deleted pertactin” as described above may also comprise or be derived from one or more parts or fragments of a pertactin, but preferably only as long as the resulting deleted pertactin still meets the requirements on sequence identity outlined above. Preferably, such a deleted pertactin comprises at least 200 amino acids, preferably at least 400 amino acids, more preferably at least 600 amino acids, and even more preferably at least 800 amino acids.

Also, to provide a deleted pertactin according to this aspect of the invention, it may be possible to replace one or more of the amino acids that are deleted to provide the deleted pertactin—i.e. as outlined above—with one or more other amino acid residues, most preferably however not such that the amino acid residues that replace the deleted amino acid residues provide essentially the same biological—e.g. immunological—effect (optionally in conjunction with the remaining amino acid residues from the pertactin sequence) as the deleted amino acid residues. Again, preferably, such a polypeptide should meet the requirements as to sequence identity outlined above, in which the amino acid residues that are substituted for the amino acid residues that are deleted to provide the deleted pertactin are not taken into account.

Preferably, any “deleted pertactin” as outlined above is such that, upon administration to a human being in a suitable manner and in a suitable amount, it is capable of eliciting in said human being an immune response. More preferably, any such deleted pertactin is such that, upon administration to a human being in a suitable manner and in a suitable amount, it is capable of eliciting in said human being a protective immune response, and in particular an immune response which protects said human being against infection by B. pertussis, and preferably also against infection by B. parapertussis, as may be determined using one or more of the techniques referred to above. Also, any such deleted pertactin is preferably such that, upon administration to a human being in a suitable manner and in a suitable amount, it is capable of eliciting in said human being a “detectable immune response”, which for an analogue may also include the generation against antibodies against the specific analogue used, and as again may be d tried using one or more of the techniques referred to above.

A deleted pertactin as outlined above may be provided in a manner known per se, such as expression of a nucleotide sequence encoding such a deleted pertactin in a suitable host organism—e.g. under the control of a suitable promoter operable in the host organism used—followed by isolation and optionally further purification of the fusion from the host organism and/or the culture medium. Again, suitable techniques for providing nucleotide sequences encoding such deleted pertactin, for (heterologous) expression thereof in a host organism, suitable host organisms (such as Escherichia coil or B. (para)pertussis) and promoters operable therein (such as T7 RNA polymerase promoter, lac promoter, or promoters derived from B. (para)pertussis, such as the promoter for Omp or filamentous hemagglutinin), and techniques for isolating the deleted pertactin(s) will be clear to the skilled person.

The deleted pertactins mentioned above may again be used as antigenic components for use in providing/formulating pharmaceutical compositions, and in particular in providing/formulating vaccines—i.e. against B. (para)pertussis—essentially in the manner outlined above for the peptides of the invention. In such applications, the deleted pertains may provide the same advantages as described above for (the vaccines containing) the peptides of the invention. In particular, as with the peptides of the invention, the deleted pertactins of the invention may circumvent the mechanisms evolved by the bacteria to direct the immune response towards conformational, variable, epitopes, and thus provide cross-immunity, also against (strains of) B. (para)pertussis.

The invention also relates to antibodies generated against a deleted pertactin as outlined above, and to pharmaceutical compositions containing such antibodies. Such antibodies and such compositions may be obtained and/or used essentially as outlined above for the antibodies against the peptides of the invention.

In yet another aspect, the invention relates to a pharmaceutical composition, and in particular to a vaccine—i.e. against B. (para)pertussis—that comprises at least one peptide of the invention in combination with at least one deleted pertactin as described above. Such pharmaceutical compositions/vaccines may also comprise one or more of further constituents—e.g. those outlined hereinabove for the vaccines of the invention—to provide a monovalent and/or polyvalent vaccine.

Although the invention has been described hereinabove mainly with respect to human applications, it should be noted that, in yet another aspect, the peptides of the invention, the deleted pertactins of the invention and/or the antibodies described above may also be used (to provide a composition suitable and/or intended) for veterinary purposes, and in particular to provide veterinary vaccines against infections by B. parapertussis and B. bronchiseptica in mammals such as cats, dogs, pigs, cattle. The preparation and use of such vaccines, which again may be monovalent vaccines or polyvalent, will be clear to the skilled person, also taking into account the further disclosure herein.

### DESCRIPTION OF THE FIGURES

The invention will now be illustrated by means of the following non-limiting Examples, and by means of the non-limiting Figures, in which:

FIG. 1 is a schematic drawing showing the structure of the pertactin gene, and the location of polymorphic sites. The proteolytic cleavage sites are indicated with asterisks and the parts of the gene resulting in the products P.69 and P.30 are indicated. The RGD sequence involved in attachment to host receptors has been shaded Note the presence of 3 types of repeats GGavP, GGfgP and GGgvP in region 1. Dashes indicate gaps in the sequence introduced to increase the number of matches. Numbers indicate the position of amino acids in Prn1 relative to the N-terminus of the unprocessed molecule.

FIGS. 2A and 2B are tables showing the amino acid sequence of peptides used for epitope-mapping and vaccination. (A) Epitope mapping of monoclonal antibodies specific for region 1 of pertactin. The sequence of each individual peptide is given, the RGD motive, involved in adherence, has been shaded. Binding of the monoclonal antibodies to the peptides is indicated by “−” (no binding) and “+” (binding). Peptides used for vaccination were conjugated to tetanus toxoid. The meningococcal peptides were used as negative controls in the vaccination experiments. (B) Epitopes bound by monoclonal antibodies. Shading in the region 1 sequence indicates epitopes bound by each individual monoclonal antibody.

FIGS. 3A and 3B represent immunoblotting gels showing differential binding of monoclonal antibodies to distinct pertactin types. Cell lysates from B. pertussis strains (expressing Prn1-6), B. bronchiseptica (Bb) and B. parapertussis (Bpp) were analyzed by inmunoblotting, using anti-pertactin monoclonal antibodies PeM5, (panel A) or PeM7, panel B). The numbers above the lanes refer to the pertactin type. Position of molecular weight markers are indicated.

FIG. 4A represents and immunoblotting gel, and FIG. 4b is a graph, showing that region 1 of pertactin is immunogenic in mice. Mice were immunized with Prn1 and the ability of the antiserun to bind to MBP fusion proteins with region 1 derived from Prn1 to Prn6 was assessed. (A) Immunoblot with anti-Prn1 mouse serum. Only the result obtained with MBP-Prn1 is shown. Identical results were obtained with MBP-Prn2 to MBP-Pr5. Equal amounts of MBP and MBP-Prn1 were loaded on the gel. Positions of pertactin and MBP-Prn1 are indicated. (B) ELISA with anti-Prn1 mouse serum. Antibody titers in serum to MBP fusion proteins, and the sequence of the pertactin region fused to MBP, are shown.

FIG. 5 is a graph showing that serum from pertussis patients harbors antibodies directed against region 1. ELISA plates were coated with Prn1. Binding of monoclonal antibodies PeM7 (directed against region 1) or PeM2 (directed against an epitope present in all pertactin variants) was allowed after pre-incubation with two-fold dilutions of human serum. Human serum was from one pertussis patient, similar results were obtained with a pool of sera from different pertussis patients.

FIGS. 6A-6D are graphs showing that immunization with peptides derived from pertactin region 1 protects against intranasal B. pertussis infection. Mice were immunized with PBS, a meningococcal peptide, a mixture of peptides derived from region 1, or with pertactin (Prn1). Peptides were conjugated to tetanus toxoid. After immunization, mice were intranasally infected with B. pertussis stain B213 expressing Prn1 . The amount of bacteria in lungs and trachea, and antibody titers, were determined 3 days post-infection. (A) Anti-tetanus toxoid IgG titers. (B) Anti-Prn1 IgG titers. (C) CFU in the lungs. (D) CFU in trachea. The thin line indicates the mean. P values are indicated. The experiment was performed three times and a representative result is shown.

FIG. 7 is a graph showing the passive protection against B. pertussis infection by a monoclonal antibody directed against region 1 of pertactin. Mice were immunized passively by administration PeM4, a monoclonal antibody directed against mumps virus, or PBS, respectively. After immunization, mice were intranasally infected with B. pertussis strain B213 (Prn1). The amount of bacteria in lungs and trachea, was determined 3 days post-infection. The thin line indicates the mean. P values are indicated. The experiment was performed three times and a representative result is shown.

FIG. 8 is a graph showing that a monoclonal antibody (PeM4) against a conserved epitope in region 1 confers cross-immunity against B. pertussis strains with distinct pertactin variants. The experiment was performed twice, and a representative result is shown See the legend to FIG. 7 for experimental details.

FIG. 9 is a graph showing protection against intranasal B. pertussis infection by immunization with peptides derived from pertactin region 1. Mice were immunized with PBS, a meningococcal peptide (control), peptide GGFGP-GGFGP-GGFGP—, peptide GGAVP-GGFGP-GGFGP, or with pertactin (Prn1). Peptides were conjugated to tetanus toxoid. After immunization, mice were intranasally infected with B. pertussis strain B213 expressing Prn1. The amount of bacteria in lungs and trachea was determined 3 days post-infection.

### EXAMPLES

### Example 1

1. Methods

1A Bacterial strain and plasmids: B. pertussis, B. parapertussis and B. bronchiseptica strains used in this study are indicated in Table I. B. strains were grown on Bordet-Gengou (BG) agar (Difco Catalog no.0048-17-5, Detroit, USA) supplemented with 1% glycerol and 15% sheep blood at 35° C. for 3 days. Escherichia coil strains DH5α and BL21 (DE3) were used for the propagation of plasmids. E. coli strains were routinely grown in L-broth or on L-agar supplemented with antibiotics. pMAL-c2 vector was purchased from New England Biolabs, Beverly, Mass., USA.

1B. DNA sequencing: The pertactin gene of all strains used in this study were sequenced previously or during this study (vide Li et al.; Mastrantonio et al.; Mooi et al.(1998) and Mooi et al. (1999), all supra; vide also Table I). DNA was isolated using standard procedures. DNA sequencing was performed by direct sequencing of PCR products. Sequencing was carried out using the ABI prism dye terminator cycle sequencing ready kit (Perkin Elmer-Applied Biosystems, Foster City, Calif., USA) and the products were analyzed on a 373 ABI DNA sequencer (Perkin Elmer).

1C. Construction of maltose binding protein containing the pertactin polymorphic region 1: Part of the open reading frame of the prn1 gene, coding for the amino acid sequence of SEQ ID NO: 1 (defined as region 1)

TIRRGDAPAGGAVPGGAVPGGAVPGGFGPGGFGPVL (SEQ ID NO: 1)

was expressed in E. coli as a fusion with the maltose binding protein (MBP) by cloning the corresponding pertactin DNA sequence in the pMAL-c2 vector (New England Biolabs, Beverly, USA). The pertactin sequence was obtained by PCR with the following two primers:

| | |
|---|---|
| 5′-CGGGATCCACGATACGGCGCGGGGAC-3′; | (SEQ ID NO:7) |
| 5′-GCTCTAGAGAGGACGGGACCGGAAGCC-3′ | (SEQ ID NO:8) |

and with chromosomal DNA of B. pertussis strain B5. In the primers the BamHI and XbaI restriction sites, introduced to facilitate cloning, are underlined, whereas pertactin-derived sequences are indicated in bold. PCR products derived from prn2, prn3, prn4, and prn5 were obtained in a similar way using the appropriate s B394, B365, B705 or B1 148, respectively (Table I). Gel-purified PCR products were digested with BamHI and XbaI and subsequently ligated in BamHI-XbaI digested pMAL-c2. All plasmid constructs were checked by DNA sequencing.

1D. Purification of maltose binding protein-pertactin fusion proteins: 500 ml E. coli cultures (BL21) carrying pMAL-c2 derivatives were grown in L-broth medium containing ampicillin (200 μg/ml) and 1% glucose at 37° C. At an OD₆₀₀=0.5 expression of the MBP-fusion protein was induced by the addition of isopropyl-β-D-thiogalactopyranoside (IPTG) to a final concentration of 0.3 mM. After induction, growth was continued for 2 hrs and cells were harvested by centrifugation at 13000×g for 20 minutes. The pellet was resuspended in lysis buffer (=10 mM NaPO₄, pH 7.2, 0.5 M NaCl, 0.25% Tween 20, 10 mM EDTA and 0.1 mM PMSF), and stored at −20° C. overnight. After thawing, the cell suspension was sonicated in ice for 4×30 seconds with 2 minutes interval (Branson Sonifier 250, normal tip, 50% output). Extracts were diluted to 100 ml with column buffer A (20 mM Tris, 200 mM NaCl, 1 mM EDTA) and applied on a 5 ml amylose column. After extensive washing with buffer A, bound proteins were eluted with buffer A, supplemented with 10 mM Maltose, and fractions of 1 ml were collected Purity was >99%, as estimated by SDS-PAGE followed by Coomassie Brilliant Blue (Serva) staining.

1E. Production of polyclonal and monoclonal antibodies: Polyclonal antiserum against pertactin was prepared by repeated injection (on day 0 and day 28) of BALB/c mice with 5 μg Prn1 kindly provided by Chiron-Biocine SpA (Siena, Italy) in 0.35% Alhydrogel in PBS. Mice were bled on day 42. Monoclonal antibodies PeM1, PeM2, PeM3, PeM4, PeM5, PeM6 and PeM7 were generated by injection of BALB/c mice subcutaneously three times with purified pertactin mixed with Specol. PeM70, PeM71 and PeM72 were generated similarly but instead of Prn1, MBP-Prn1 was used as the antigen. For the production of PeM68, mice were injected with MBP-Pm5. PeM80, PeM84 and PeM85 were generated using Prn5. Three days before the fusion, mice were boosted intravenously. Spleens cells were fused with mouse myeloma cells SP2/0 using 50% PEG 1500 (Boehringer, Mannheim, Germany). Hybridomas secreting antibody to pertactin were selected by ELISA and cloned twice by limiting dilution. Monoclonal antibodies were purified by protein-G affinity chromatography (Pharmacia, Uppsala, Sweden).

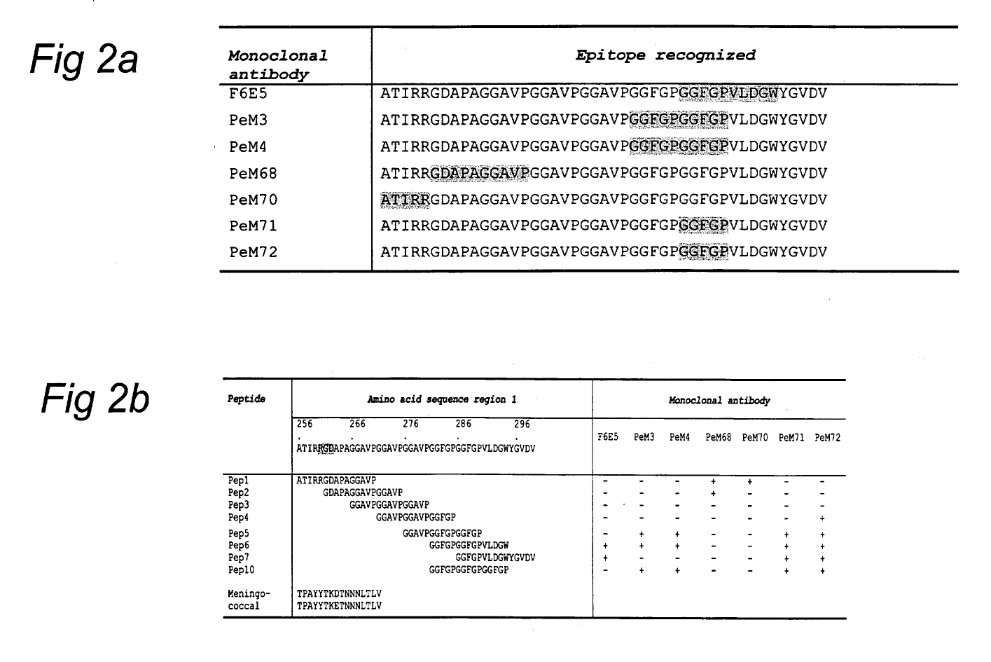

1F. Synthesis of Deptides: Peptides were assembled by using an automated multiple peptide synthesizer, equipped with a 48-column reaction block (AMS 422, ABIMED Analysen-Technik Gmbh, Langenfeld, Germany), as described by Brugghe et al., (1994), Int. J. Peptide Protein Res. 43:166-172. Peptides used for epitope mapping were N-terminally acetylated multiple (i.e. octameric) antigenic peptides (MAPs), prepared as described previously Rouppe van der Voort et al., FEMS Immunol. Med. Microbiol. (1997) 17:139-148). The amino acid sequences of the pertactin region 1 specific peptides are listed in FIG. 2a Peptides used for immunization contained the same amino acid sequence but were monomeric. These peptides were N-terminally elongated with an S-acetylmercaptoacetyl group (Drijfhout et al., Anal. Biochem. (1990) 187:349-354) and conjugated to tetanus toxoid (Brugghe et al., (1994), Int. J. Peptide Protein Res. 43:166-172; and van der Ley et al., Infect Immun. (1991) 59:2963-2971). Control peptide-tetanus toxoid conjugates used for immunizations were derived from meningococcal PorA.

1G. Immunoblotting: Cell-free extracts of B. pertussis (strains B391, B596, B647, B705, B935, B1120, B14 and B24), or purified MBP-fusion proteins, were analyzed by sodium dodecyl sulfite-polyacrylamide (10%) gelelectrophoresis (SDS-PAGE) as described by Laemmli, Nature (Lond.) (1970) 227:680. Proteins were transferred to nitrocellulose filters by electroblotting (Biometra semid-dry blotting) using 5 mA/cm² for 30 minutes. After blocking with 0.5% protifar non-fat dry milk 0.1% BSA and 0.1% Tween 20 in PBS, the filters were incubated for 2 hrs with antiserum or monoclonal antibodies. Filters were washed 3 times for 10 minutes in PBS containing 1% Tween (PBS-1% Tween 20) and subsequently incubated for 1 hr with goat anti-rabbit total IgG conjugated to horse-radish peroxidase (IRP) in PBS. After two 10 minutes washes in PBS supplemented with 1% Tween 20, followed by a 10 min wash irr PBS, filters were incubated with the ECL substrate (Amersbam Pharmacia Biotech) and bound HRP was visualized by exposing a sheet of autoradiography film (Hyperfilm-ECL).

1H. Epitope mapping: Polystyrene 96-well plates (Greiner ELISA) were coated for 2 h at 37° C. with 100 μl/well multimeric peptides (2 μg/ml) dissolved in PBS. Plates were washed 4 times with 200 μl PBS supplemented with 0.05% Tween 20 PABST) per well using the TitertekPlus M96V washer and blocked by incubation with PBST supplemented with 0.5% Bovine serum albumin (BSA, Sigma) for 1 hr at room temperature. Monoclonal antibodies (murine), diluted in 0.5% BSA in PBST were added to the wells and incubated for 2 h at 37° C. followed by four washings as described above. Bound antibodies were detected using HRP-conjugated anti-mouse total IgG (Cappel, Organon Technica). Mouse IgG subclasses were determined by using mouse monoclonal antibody isotyping kit (Isostrip, Boehringer). Extinctions (OD₄₅₀) were measured with a BioTek plate reader (EL312e, BioTek Instr.).

1L. Immunization and challenge of mice: B. pertussis strains B213 (Tohama) (prn1), B1296, (prn2) or B1400 prn3) were grown on BG agar supplemented with streptomycin (30 μg/ml) at 35° C. for 3 days. Subsequently the bacteria were plated on BG agar plates without streptomycin. After 3 days, bacteria were harvested and resuspended in Verwey medium (Verwey et al., J. Bacteriol. (1949) 50:127-134) to a concentration of 5×10⁸ bacteria/ml. An aliquot of the final suspension was diluted and plated to determine the CFU of the challenge inoculum. Groups of 8 female BALB/c mice (RIVM or Harlan) were used for immunization. For passive immunization, 250 μg of a purified monoclonal antibody was injected intravenously into 4 wks old mice. Mice were infected 24 h later. For active immunization, 3 wk old mice were immunized subcutaneously, on day 0, day 14 and day 28 with 0.5 ml PBS containing 20 μg Quil A (Spikoside, Iscotec AB, Lulea, Sweden) and 50 μg tetanus-conjugated peptide. When a mix of 7 peptides was used, equal amounts of each peptide (i.e. 6.25 μg) were present in the vaccine except for peptide 4 of which 12.50 μg was present. Mice were infected 14 days after the last immunization. For infection mice were lightly anesthetized with ether, and a drop of 20 μl of the inoculum was placed on top of each nostril and allowed to be inhaled by the animal. Mice were infected with a total amount of 2×10⁷ B. pertussis cells. Three days after infection mice were sacrificed by intraperitoneal injection of an overdose of barbiturate (Nembutal, Sanofi/Algin) and lungs, trachea and blood were collected. For the determination of bacterial colonization, lungs were homogenized in 900 μl Verwey medium for 10 seconds at 20.000 rpm using a handheld homogenizer (pro scientific, pro200). The trachea was excised and vortexed in 200 μl Verwey medium with 5 glass pearls (diameter 3 mm) for 30 seconds. Appropriate dilutions of the homogenates were plated on BG agar plates supplemented with streptomycin (30 μg/ml). The number of CFU was counted after 4 days of incubation at 35° C. Levels of serum antibody directed against antigens used for vaccination was determined by ELISA as described above. Plates were coated using tetanus toxoid (2 μg/ml) or Prn1 (2 μg/ml). Extinctions were measured with a BioTek plate reader (EL312e, BioTek Instr.) and titers were calculated with Kineticalc (KC4, BioLyse).

1J. Blocking ELISA: Blocking Elisa was performed essentially as described in Berbers et al., (1993) J. of Virological Methods 42:155-168. Plates were coated with Prn1 (2 μg/ml) as described above. After washing and blocking, the wells were incubated with 100 μl of serial two-fold dilutions of human serum diluted in 0.5% BSA in PBST for 2 h at 22° C. Human sera were from two sources. One serum was from a vaccinated child that was recently infected with pertussis. The serum-sample was taken 47 days after the onset of the disease. The second serum sample comprises a mix of sera from several recently infected pertussis patients, which were fully vaccinated After washing the plates, a pertactin-specific murine monoclonal antibodies PeM7, PeM2, PeM5 or PeM6 was added and the plates were incubated for 2 h at 22° C. After the plates were washed thoroughly, they were developed and read as described above.

2. Results

2A. Characterization of Monoclonal Antibodies Against Pertactin

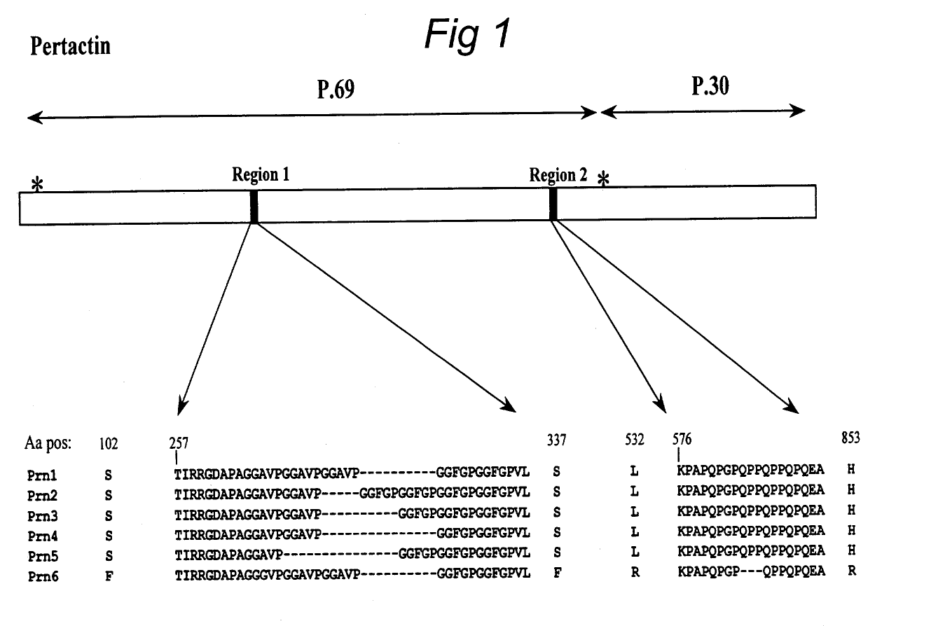

Previously, the art identified 6 different pertactin types in B. pertussis stains circulating in Europe (vide Mastantonio et al.; Mooi et al. (1998) and Mooi et al. (199), al supra). Variation between these pertactin types is mainly restricted to region 1, and consists of deletions or insertions of the repeat unit GGxxP (FIG. 1). Herein, it was investigated whether the polymorphism in region 1 affected antibody binding, i.e. represented antigenic variation. To this purpose several monoclonal antibodies, were raised using pertactin variants and MBP-fusion proteins with region 1 derived from different pertactin variants.

Binding of the monoclonal antibodies to the 6 B. pertussis pertactin types and the homologous proteins derived from the closely related species B. bronchiseptica and B. parapertussis was analyzed by immunoblotting. The amount of pertactin loaded on the gels was standardized using monoclonal antibody E4D7 which is directed against a conserved part of pertactin (i.e. APQPPAGR, which is located close to region 2) by the general method described by Tam and Zavala (1998; J. Immunol. Med. 124: 53-61). The pertactin genes of all strains used were sequenced completely to identify polymorphism outside region 1. Compared to prn1, the B. bronchiseptica and B. parapertussis pertactin genes revealed amino acid substitutions over the whole length of the gene (not shown). However, within B. pertussis only prn6 showed polymorphism outside region 1; compared to prn1, prn6 contained four amino acid substitutions at amino acid positions 102, 337, 532, 853 and a deletion of 3 amino acids in region 2 (FIG. 1). Of the 14 monoclonal antibodies tested, 7 cross-reacted with pertactin derived from B. bronchisceptica and B. parapertussis (Table 2).

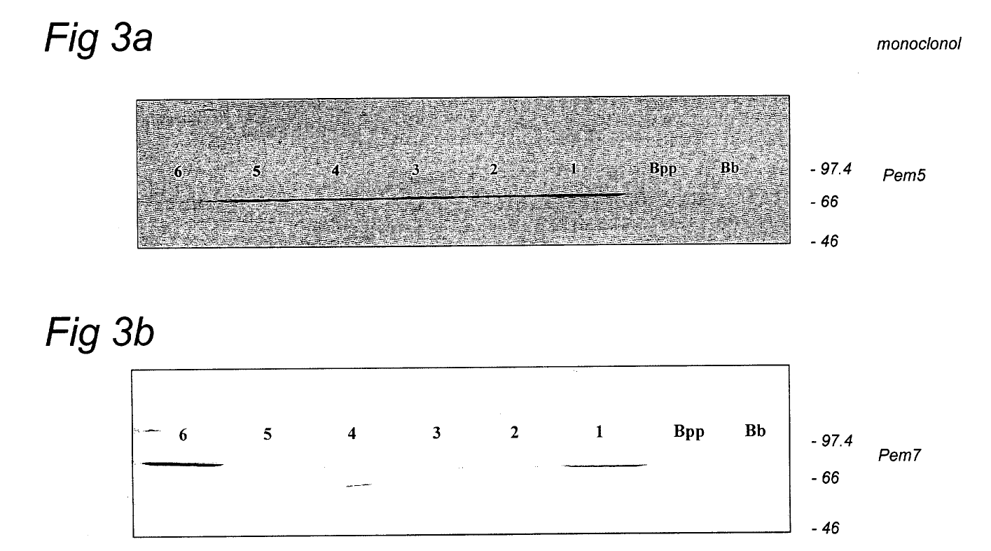

Out of the 14 monoclonal antibodies, 2 (Pem5 and PeM7) showed differential binding to B. pertussis pertactin (FIG. 3, Table 2). PeM5 showed highest and lowest binding to Prn1 and Prn6, respectively. Intermediate binding was observed with Prn2-5. This indicates that PeM5 recognizes an epitope that comprises part of region 1. In region 1, Prn1 and Prn6 differ in 1 amino acid, at position 268 (FIG. 1), implicating this residue as part of the structure of the PeM5 epitope.

PeM7, although raised with Prn1, showed a much stronger reaction with Prn6 compared to Prn1. Weak binding was observed with Prn2-5 (FIG. 3). Since, with the exception of Prn6, the different pertactin proteins do not vary outside region 1, the differential binding of monoclonal antibodies is due to recognition of an epitope encompassing region 1. PeM5 and Pem7 did not bind to pertactin from B. bronchiseptica and B. paraertussis (FIG. 3). Taken together, these results indicate that variation in region 1 affects antibody binding.

The ability of the monoclonal antibodies to bind to region 1 was also studied with MBP-fusion proteins harboring region 1 derived from the pertactin variants Prn1-5. Binding was assessed by immunoblotting. Of the 14 monoclonal antibodies tested, 7 (PeM3, PeM4, PeM68, PeM70, PeM71, PeM72 and F6ES) bound to the fusion proteins but not to MBP (Table 2). No quantitative differences were observed between these 7 monoclonal antibodies with respect to binding to the MBP-fusion proteins. The monoclonal antibodies that did not bind to the MBP fusion proteins may recognize an epitope located outside region 1. It is also possible that these monoclonal antibodies recognize a conformational epitope encompassing region 1, which folds differently in a MBP fusion protein compared to pertactin. In fact this is probably the case with PeM5 and PeM7, which do not bind to the MBP fusion proteins, although the type specificity of these monoclonal antibodies indicated that they bound to region 1 of pertactin Significantly, these data indicate that a large number (8) of all (14) monoclonal antibody raised against intact pertactin were directed against region 1, suggesting it represents an immunodominant region in mice.

2B. Epitope Mapping

To delineate the epitopes recognize by the monoclonal antibodies described above, a set of 8 overlapping peptides corresponding to the region 1 (Pep1 to 7 and Pep10) was used (FIG. 2a). The 7 monoclonal antibodies F6E5, PeM3, PeM4, PeM68, PeM70, PeM71 and PeM72) which bound to the fusion proteins also bound to the peptides (FIG. 2a). The monoclonal antibody F6E5 was previously reported to bind a pertactin peptide of 128 amino acids encompassing the RGD and the GGxxP repeat (Charles et al., Eur. J. Immunol. (1991), 21:1147-1153). Herein, it is shown that F6E5 bound to peptides 6 and 7 only, defining the sequence GGFGPVLDGW as its epitope. Based on their differential binding to the peptides the epitopes of the other monoclonal antibodies were also delineated, and are shown in FIG. 2b. The epitopes comprised of GGFGPGGFGP and GGFGP were each recognized by two monoclonal antibodies, respectively, PeM3, PeM4 and PeM71, PeM72. Epitopes for PeM68 and PeM70 were identified as GDAPAGGAVP and ATJRR, respectively. PeM5 and PeM7 did not bind to the peptides, which is consistent with the assumption that they recognize a conformational epitope.

2C. Antibodies Against Region 1 Are Induced in Mice and Humans

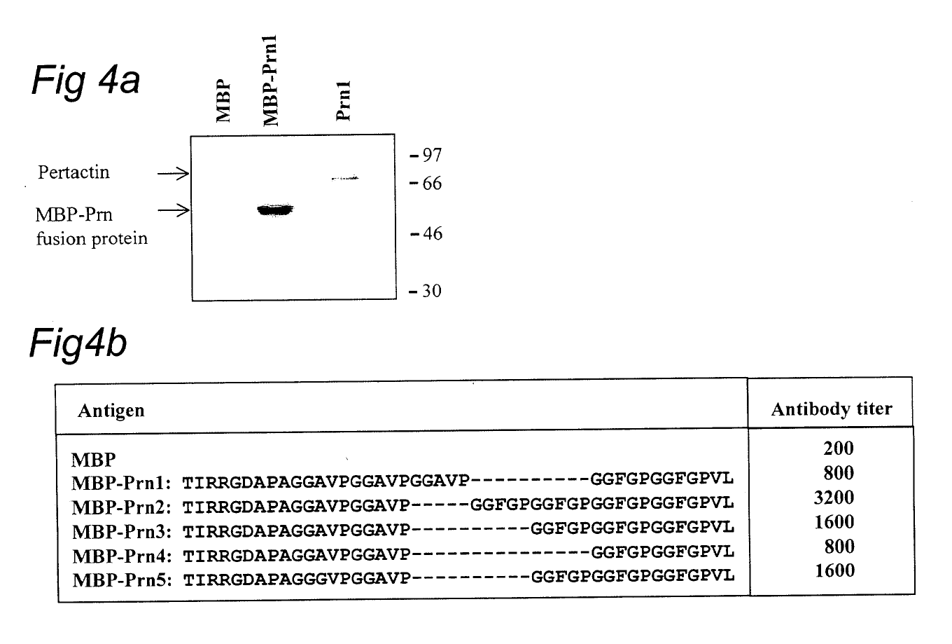

In order to further confirm that antibodies are induced against region 1 of pertactin, mice were immunized with Prn1, and the resulting antiserum was tested with immunoblotting and ELISA (FIG. 4). As antigen MBP-fusion proteins harboring region 1 from pertactin variants Prn1-5 were used. Immunoblotting detected mouse antibodies against all MBP-fusion proteins, but not MBP, indicating the presence of antibodies against region 1 (FIG. 4a). Antibody titers against the different pertactin region 1 sequences were assessed by ELISA (FIG. 4b). The results showed that, although this serum was raised against the Prn1 protein, the highest binding was observed to MBP-Prn2, whereas the lowest binding was found to MBP-Prn1 and MBP-Prn4. An intermediate level of binding was observed with MBP-Prn3 and MBP-Prn5. The level of binding correlated best with the number of GGFGP repeats (FIG. 4b), suggesting that after immunization with Prn1 a substantial fraction of the antibodies which recognize linear epitopes in region 1 were directed against this epitope.

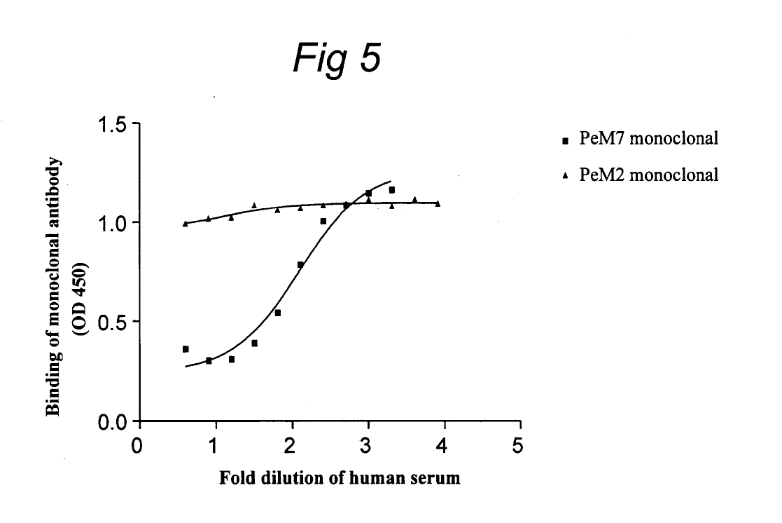

Since human sera cross-reacted with the MBP it was not possible to use the MBP fusion proteins to detect antibodies against region 1. Therefore, the presence of region 1-specific antibodies in human sera was investigated using a blocking ELISA. In this assay the ability of human antibodies to compete with monoclonal antibody PeM7, which binds to region 1 (see above), for binding to immobilized Prn1 was assessed. The serum sample was from a child recently infected with B. pertussis. Similar results were obtained with pooled sera derived from several children (not shown). Binding of PeM7 to Prn1 was inhibited in a dose-dependent manner (FIG. 5), indicating the presence of human antibodies directed against region 1. Binding of PeM2, which recognizes an epitope present in all tested pertactin variants (Table 2) was not inhibited by human antibodies (FIG. 5). The monoclonal antibody PeM5 is similar to Pem7 in that it binds to a conformational epitope encompassing region 1 (see above), also competed with human antibodies (not shown). Interestingly, monoclonal antibodies which recognized linear epitopes (e.g. PeM4 and F6E5) in region 1 were not able to compete with human serum for binding to pertactin (not shown). These results indicate hat, in its native state, region 1 elicits antibodies in mice and humans.

2D. Peptides Derived From Pertactin Region 1 Elicit Protective Immunity

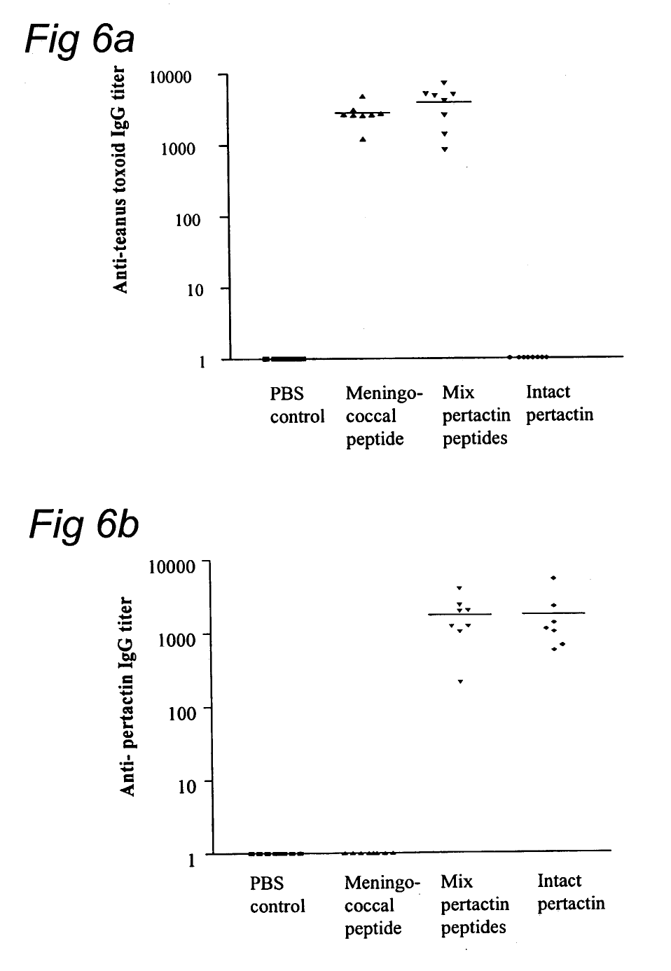

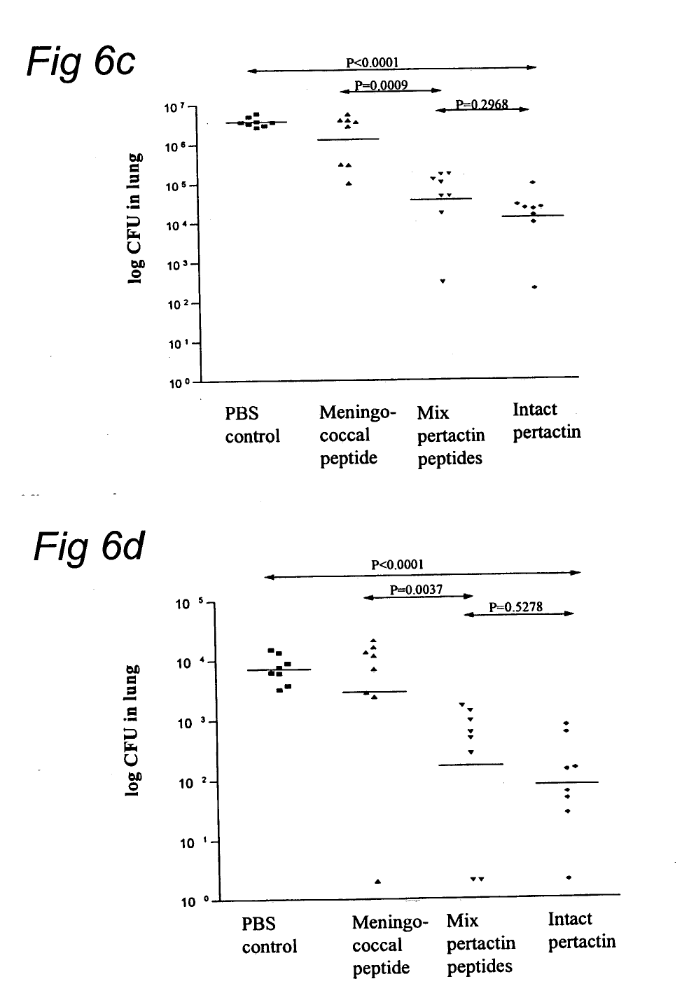

To determine whether region 1 was able to induce a protective immune response, mice were immunized with a mixture of seven overlapping peptides derived from region 1 (pep1 to pep7, FIG. 2a). Mice in the control group were immunized with a meningococcal peptide (FIG. 2a) or with PBS. Immunization with Prn1 served as a positive control. Both pertain peptides and meningococcal peptides were conjugated to tetanus toxoid. Analysis of the serum samples of immunized and infected mice revealed that high antibody titers to tetanus toxoid were observed in both groups immunized with the peptides (FIG. 6a), while only mice immunized with the pertactin peptides or with the intact pertain had anti-pertactin titers (FIG. 6b). Mice immunized with the pertactin peptides had up to 35-fold less bacteria in their lungs compared to the group immunized with the meningococcal peptide (P=0.0009, FIG. 6c). The level of protection induced by the pertactin peptides was 6-fold less than that achieved by intact pertactin (P=0.2968). Immunization with pertactin peptides reduced colonization in the trachea 16-fold compared to mice immunized with the meningococcal peptide (P=0.0037, FIG. 6d). As was observed for the lungs, immunization with pertain peptides was somewhat less (3-fold) effective compared to immunization with native pertactin (P=0.5278, FIG. 6d). In conclusion, both in the trachea and lungs, peptides derived from region 1 were able to induce protective immunity.

2E. Passive Immunization With a Monoclonal Antibody Against Region 1 Confers Protection

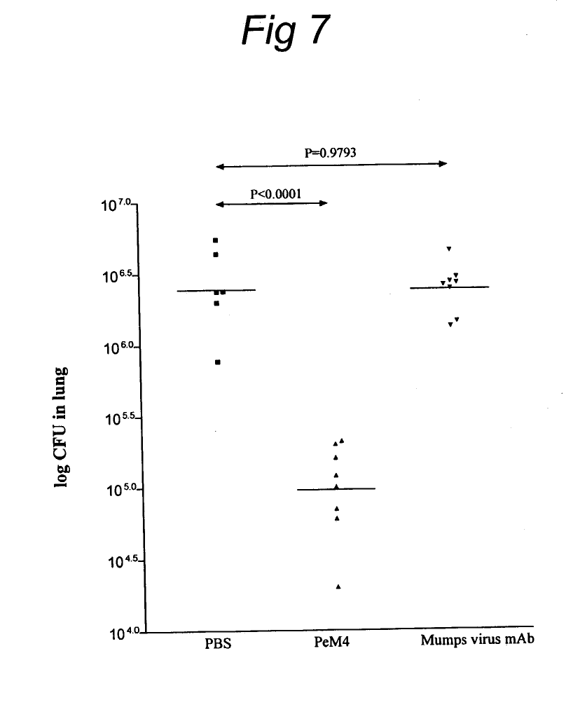

It was investigated whether a monoclonal antibody directed against region 1 was also able to protect against infection PeM4, which binds to the GGFGPGGFGP sequence in region 1 (see above), was administered intravenously 24 h prior to challenge. Control mice were injected with a monoclonal antibody directed against the mumps virus or with PBS. Nice injected with PeM4 showed over 10-fold less colonization of the lungs by B. pertussis, compared to mice injected with PBS (P<0.0001) (FIG. 7). Passive immunization with the mumps virus monoclonal antibody failed to reduce B. pertussis colonization of the lungs comparable to the PBS control. In the trachea the reduction in colonization by PeM4 was less pronounced, in some experiments an approximately two-fold drop (P=0.068) in colonization was seen whereas in others no significant drop in colonization was observed (not shown) Taken together, these results confirm that the immune response directed to pertactin region 1 contributes to protection in the lungs.

2F. A Monoclonal Antibody Against a Conserved Linear Epitope in Region 1 Confers Cross-Protection Against Strains With Different Pertactin Variants

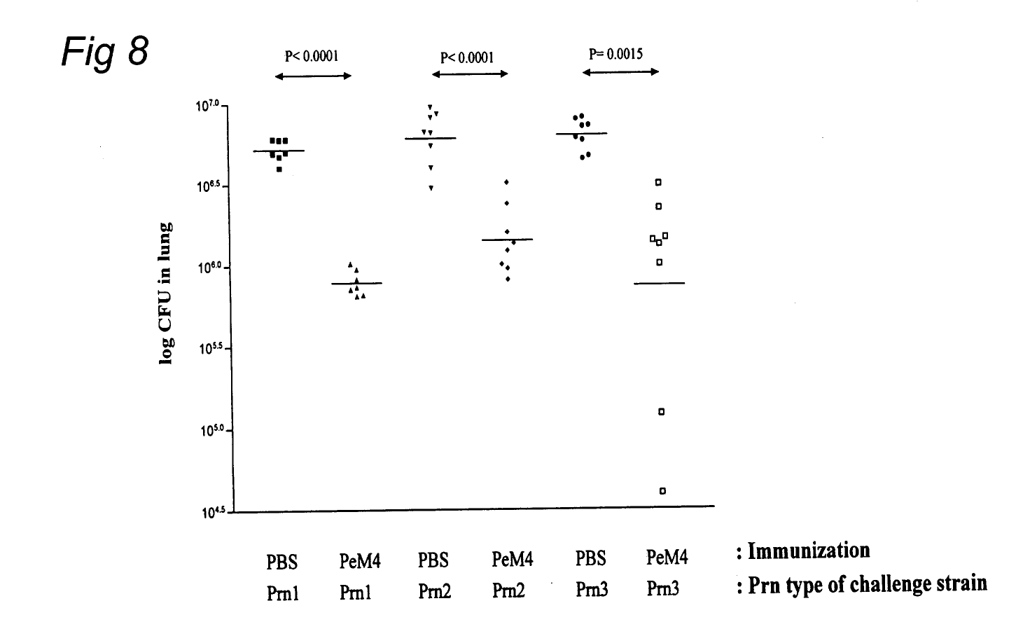

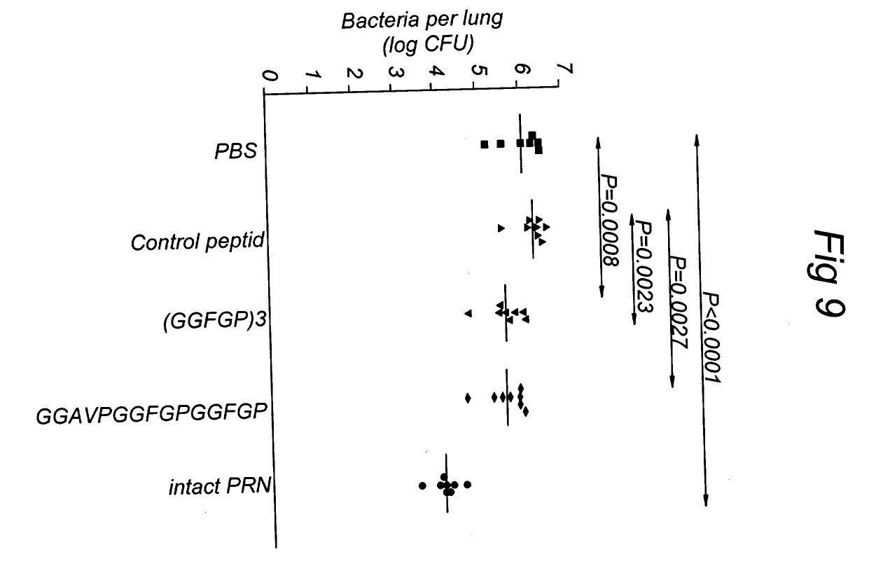

Since the monoclonal antibody PeM4 recognizes an epitope present in all known B. pertussis variants of pertactin (FIGS. 1 and 2b) it was determined whether PeM4 was able to protect against stains which differed in the type of pertactin expressed. Mice were passively immunized with PeM4 24 h before challenge with B. pertussis strain B213 (Prn1), B1296 (Prn2) or B1400 (Prn3). Control mice were injected with PBS before challenge. PeM4 significantly (P≦0.0015) reduced the colonization of B. pertussis stains expressing Prn1, Prn2 and Prn3 in the lungs compared to the PBS controls (FIG. 8), showing that it is possible to elicit antibodies that protect equally against strains expressing distinct pertactin variants.

### Example 2

Immunization of Mice With The Peptide GGAVP-GGFGP-GGFGP

The sequence GGAVP-GGFGP-GGFGP is found in region 1 of all pertactin variants identified to date. Mice were immunized with his peptide to determine if it conferred protection against B. pertussis. As indicated in the figure, mice immunized with GGAVP-GGFGP-GGFGP were significantly less colonized in the lungs compared to mice vaccinated with a control (meningococcal) peptide (P=0.0027). A second peptide GGFGP-GGFGP-GGFGP) found in region 1 of the 2 predominant pertactin types in The Netherlands, Finland and Italy (i.e. pertactin type 2 and 3) also conferred significant protection compared to the control peptide (P=0.0023). These results provide additional evidence that conserved sequences from region 1 can induce cross-protective antibodies.

TABLE 1
Bordetella strains used in this application.

| | original | | | prn | Accession |
|---|---|---|---|---|---|
| Strain | designation | species | Source | type | number |
| B5^{a} | | B. pertissus | US | prn1 | AJ011091 |
| B391^{b} | | B. pertussis | Netherlands | prn1 | AJ011091 |
| B394^{a} | | B. pertussis | Netherlands | prn2 | AJ011092 |
| B596^{b,a} | | B. pertussis | Netherlands | prn2 | AJ011092 |
| B365^{b} | | B. pertussis | Netherlands | prn3 | AJ011093 |
| B647^{b} | | B. pertussis | Netherlands | prn3 | AJ011093 |
| B705^{b,a} | | B. pertussis | Finland | prn4 | AJ011015 |
| B935^{b} | | B. pertussis | Italy | prn5 | AJ011016 |
| B1148^{a} | | B. pertussis | Italy | prn5 | AJ011016 |
| B1120^{b} | 18323 | B. pertussis | US | prn6 | AJ132095 |
| B213^{c} | Tohama | B. pertussis | Japan | prn1 | AJ011091 |
| B1296^{c} | | B. pertussis | Netherlands | prn2 | AJ011092 |
| B1400^{c} | | B. pertussis | Netherlands | prn3 | AJ011093 |
| B14^{b} | | B. bronchisep- | | prn | AJ245927 |
| | | tica | | | |
| B24^{b} | | B. paraper- | Netherlands | prn | X54547 |
| | | tussis | | | |

^{a}Used for construction of the MBP-fusion proteins
^{b}Used for immunoblotting.
^{c}Used for challenge experiments.
^{d}Accession number refers to an identical, previously submitted, sequence from another strain.

TABLE 2
Reactivity of monoclonal antibodies with pertactin variants or
MBP-pertactin fusion proteins containing region 1.

| | | Antigens used for immunoblotting | | | | | | | | | |
|---|---|---|---|---|---|---|---|---|---|---|---|
| Monoclonal | Antigen used for | | | | | | | | | MBP- | |
| antibody | immunization | Pm1 | Pm2 | Pm3 | Pm4 | Pm5 | Pm6 | Bbr | Bpp | Pm1-5^{a} | MBP |
| PeM 1 | Pm1 | + | + | + | + | + | + | + | + | − | − |
| PeM 2 | Pm1 | + | + | + | + | + | + | + | + | − | − |
| PeM 3 | Pm1 | + | + | + | + | + | + | − | − | + | − |
| PeM 4 | Pm1 | + | + | + | + | + | + | − | − | + | − |
| PeM 5 | Pm1 | + + | + | + | + | +/− | +/− | − | − | − | − |
| PeM 7 | Pm1 | + | +/− | +/− | +/− | +/− | + + | − | − | − | − |
| PeM 68 | MBP-Pm5 | + | + | + | + | + | + | + | + | + | − |
| PeM 70 | MBP-Pm1 | + | + | + | + | + | + | + | +/− | + | − |
| PeM 7l | MBP-Pm1 | + | + | + | + | + | +/− | + | +/− | + | − |
| PeM 72 | MBP-Pm1 | + | + | + | + | + | + | + | + | + | − |
| PeM 80 | Pm5 | + | + | + | + | + | + | − | − | − | − |
| PeM 84 | Pm5 | + | + | + | + | + | + | + | + | − | − |
| PeM 85 | Pm5 | + | + | + | + | + | + | − | − | − | − |
| F6E5 | WCV | + | + | + | + | + | + | − | − | + | − |

^{a}Identical results were obtained with MBP-Pm1 to MBP-Pm5.

1. A peptide consisting of 6 and 40 contiguous amino acid of the amino acid sequence of “region 1” of a B, pertussis pertactin, or an analogue of the polypeptide.

2. A peptide according to claim 1, whereby the peptide consists of between 10 and 15 contiguous amino acid residues from the amino acid sequence of “region 1” of a B. pertussis pertactin, or an analogue of the peptide.

3. A peptide according to claim 1, whereby the peptide consists of between 6 and 36 contiguous amino acid residues from the amino acid sequence of SEQ D NO: 1, or an analogue of the peptide.

4. A peptide according to claim 3, whereby the peptide consists of between 10 and 15 contiguous amino acid residues from the ammo acid sequence of SEQ ID NO: 1, or an analogue of the peptide.

5. A peptide consisting of one or more repeats GGFGP or one or more repeats GQAVP, in which the total number of such repeats is between 2 and 10, or an analogue of the peptide.

6. A peptide according to any one of the preceding claims, whereby the peptide is characterized in that it provides a protection against B. pertussis, B. parapertussis, or both, which is at least 10% of the protection as provided by the polypeptide of SEQ ID NO: 5, or an analogue of the peptide.

7. A peptide according to any one of the preceding claims, the peptide comprising the amino acid sequence of SEQ ID NO: 5, or an analogue of the peptide.

8. A peptide according to any one of the preceding claims, the peptide having a length of between 6 and 100 amino acids.

9. A polypeptide comprising an amino acid sequence of a peptide as defined in any one of claims 1-8, whereby the amino acid sequence of the peptide is linked to at least one further amino acid sequence which is different from the amino acid sequence with which the peptide is natively associated in a naturally occurring pertactin.

10. A polypeptide with the amino acid sequence of a pertactin, the polypeptide being characterized in that one or more amino acids that are part of “region 1” or “region 2” have been removed

11. A polypeptide according to claim 10, the polypeptide being characterized in that “region 1” or “region 2” have essentially completely been removed.

12. A polypeptide according to claim 10, the polypeptide having the amino acid sequence of SEQ ID NO: 6, or the amino acid sequence of a naturally occurring or synthetic analogue or variant thereof, the polypeptide being further characterized in that:
a) at least 5 of the amino acid residues at positions 266-290 have been removed; or
b) at least 3 of the amino acid residues at positions 569-603 have been removed.

13. A polypeptide according to claim 12, the polypeptide being characterized in that:
a) all of the amino acid residues at positions 266-290 have been removed; or
b) all of the amino acid residues at positions 569-603 have been removed.

14. A polypeptide according to claim 12, the polypeptide being characterized in that:
a) from the amino acid sequence between positions 260 and 290 of SEQ ID NO: 6, at least one “GGAVP” or “GGFGP” repeat has been removed; or
b) from the amino acid sequence between positions 569 and 603 of SEQ ID NO: 6,at least one “PQP” motif has been removed.

15. A polypeptide according to claim 14, the polypeptide being characterized in that:
a) all “GGAVP” or “GGFGP” repeats have been removed; or
b) all “PQP” motifs have been removed.

16. A polypeptide according to claim 12, the polypeptide being characterized in that it lacks at leas one of the amino acid sequences selected from the group consisting of
a) the sequence from position 266 to position 290;
b) the sequence from position 270 to position 285;
c) the sequence from position 569 to position 603;
d) the sequence from position 572 to position 599;
e) the sequence from position 575 to position 596;
f) the sequence from position 578 to position 594;
g) the sequence from position 531 to position 591; and
h) the sequence from position 584 to position 588.

17. A composition comprising a peptide or a polypeptide as defined in any one of the preceding clams, whereby the composition further comprises one or more components selected from the group consisting of
a) a pharmaceutically acceptable carrier or excipients;
b) an immunological adjuvant;
c) a further pertussis or parapertussis antigen; and
d) an antigen that, upon administration to a human being, protects a human or animal against one or more infectious diseases other than pertussis.

18. Use of a peptide or a polypeptide according to any of claims 1-16 in the preparation of a vaccine against B. pertussis, B. parapertussis, B. bronchiseptica, or combinations thereof with one or more further infectious diseases of mammals.

19. An antibody, generated against a peptide or a polypeptide according to any of claims 1-16.

20. A composition comprising an antibody as defined in claim 19, whereby the composition further comprises one or more components selected from the group consisting of:
a) a pharmaceutically acceptable carrier or excipients;
b) an immunological adjuvant;
c) a further pertussis or parapertussis antigen; and
d) an antigen that, upon administration to a human being, protects a human or animal against one or more infectious diseases other than pertussis.
